(19) United States
(12) Patent Application Publication
(10) Pub. No.: US 2003/0215101 A1
(43) Pub. Date: Nov. 20, 2003

(54) Active noise control system with an elongated transmission member

(75) Inventors: Philip Edward Arthur Stuart, Chatham (CA); Claudette Kennette, Windsor (CA); David S. Marion, Belle River (CA); Richard McWilliam, Shedden (CA)

Correspondence Address: SIEMENS CORPORATION, INTELLECTUAL PROPERTY LAW DEPARTMENT, 170 WOOD AVENUE SOUTH, ISELIN, NJ, 08830, US

(73) Assignee: Siemens VDO Automotive, Inc.

(21) Appl. No.: 10/304,278

(22) Filed: Nov. 26, 2002

Related U.S. Application Data
(60) Provisional application No. 60/380,706, filed on May 15, 2002.

(51) Int. Cl. A61F 11/06; G10K 11/16; H03B 29/00

(52) U.S. Cl. 381/71.4

(57) ABSTRACT

An active noise control system includes a speaker located within an enclosure within a vehicle body component. The speaker enclosure is typically located forward of a vehicle wheel well. An elongated transmission member communicates canceling noise from the speaker enclosure over the wheel well to an outlet adjacent the firewall. The elongated transmission member focuses and directs cancellation noise toward the firewall and bypasses the wheel well and any restriction created thereby.

The present application claims priority to U.S. Provisional Patent Application Serial No. 60/380,706, filed May 15, 2002.

### BACKGROUND OF THE INVENTION

The present invention relates to a vehicle noise cancellation system, and more particularly to an active noise control that communicates cancellation noise toward a passenger compartment.

Manufacturers have employed active and passive methods to reduce engine noise within the passenger compartment. Such noise frequently emanates from the engine, travels through the air induction system and emanates into the passenger compartment.

Active noise cancellation (ANC) systems typically utilize a speaker to create a canceling noise that attenuates engine noise. The noise created is out of phase with the engine noise and combines with this noise to result in its reduction. Generally, this noise is generated in proximity to the inlet of the air induction system. In one such system, a control unit, such as a digital signal processor, obtains data from the vehicle engine, creates a predictive model of engine noise, and thereby generates the appropriate cancellation signal based on the results of this model. This signal is then transmitted to the speaker, which transforms this signal into a canceling noise. Because the control unit may not perfectly model engine noise, an error microphone is placed in proximity to the intake of the air induction system to determine if engine noise need be further attenuated.

Although effective, the speaker may be displaced a relatively great distance from the passenger compartment. A relatively large speaker is typically provided to assure sufficient canceling noise is generated to reach the passenger compartment. Furthermore, the cancellation noise is often somewhat attenuated prior to reaching the passenger compartment. The speaker may require additional packaging space which may be unavailable.

Accordingly, it is desirable to provide an effective ANC system which requires minimum packaging space yet effectively communicates canceling noise to the vehicle passenger compartment.

### SUMMARY OF THE INVENTION

The active noise control system according to the present invention includes a speaker located within an enclosure within a vehicle body component. The speaker enclosure is typically located forward of a vehicle wheel well. An elongated transmission member communicates canceling noise from the speaker enclosure over the wheel well to an outlet adjacent the firewall. The elongated transmission member focuses and directs cancellation noise toward the firewall and bypasses the wheel well and any restriction created thereby.

The elongated transmission member is a tubular member having a flared outlet. The outlet is located a distance from a firewall adjacent a vehicle passenger compartment. That is, the outlet need not penetrate the firewall. Installation is thereby simplified and no structural weakness is introduced.

Alternatively, the elongated transmission member may contact the firewall or pass therethrough Furthermore, the elongated transmission member may alternatively be a solid member that transmits cancellation noise through resonance thereof. The elongated transmission member may alternatively be a hollow member having a closed end or be closed with a flexible membrane to amplify the cancellation noise through resonance thereof.

The present invention therefore provides an effective ANC system which requires minimum packaging space and effectively communicates canceling noise to the vehicle passenger compartment.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 is a schematic view of an active noise cancellation (ANC) system;

FIG. 2 is general schematic view of a vehicle body component having the ANC system mounted therein;

FIG. 3 is a sectional view of the vehicle body component taken along line 3-3 of FIG. 2;

FIG. 4 is an expanded view of one end of an elongated transmission member;

FIG. 5 is an expanded view of one end of another elongated transmission member;

FIG. 6 is an expanded view of one end of another elongated transmission member;

FIG. 7 is an expanded view of one end of another elongated transmission member; and

FIG. 8 is an expanded view of one end of another elongated transmission member.

### DETAILED DESCRIPTION OF THE PREFERRED EMBODIMENT

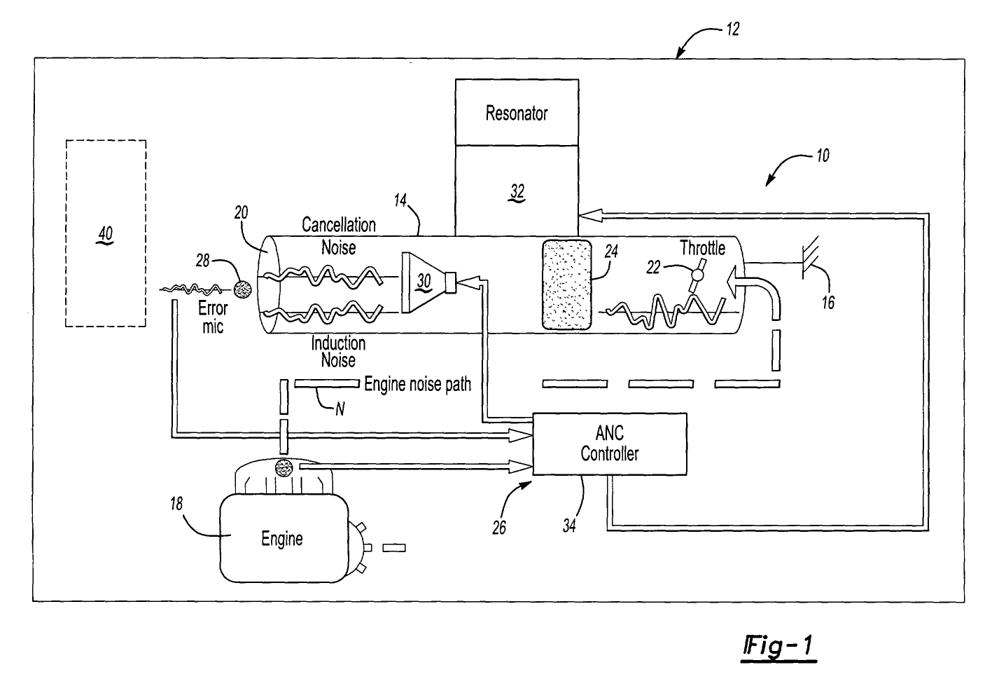

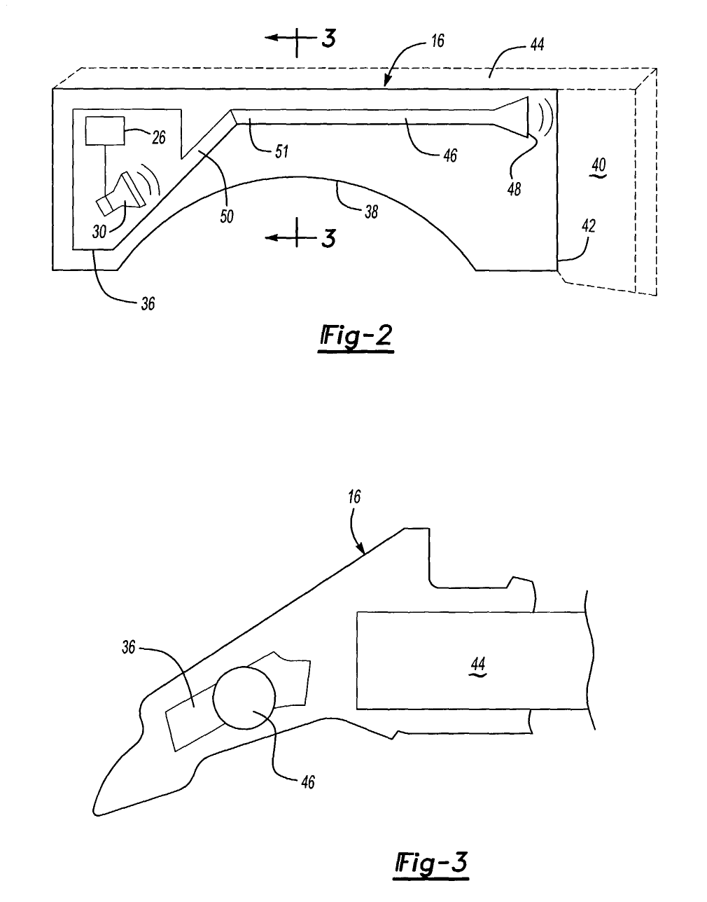

FIG. 1 illustrates a general schematic view of an air introduction system 10 for a vehicle 12. The air introduction system 10 includes an air introduction body 14 mounted adjacent a vehicle body component 16 (also illustrated in FIG. 2) such as a vehicle bumper. The air introduction body 14 intakes ambient air and communicate airflow to an engine 18. As known, noise and vibration (illustrated schematically at N) from the engine 18 emanates through the air introduction system 10. The air introduction body 14 includes an inlet 20 which communicates with ambient atmosphere through a throttle 22 and an air filter 24. It should be understood that the illustrated embodiment is schematic and that other arrangements will also benefit from the present invention.

An active noise control system 26 is mounted adjacent the air introduction body 14 to control engine noise which is radiated through the air introduction system 10. The active noise control system 26 preferably includes an error microphone 28, a speaker 30 and an active resonator 32 which communicate with an ANC controller 34. The error microphone 28 is located in a reception area such that the speaker 30 of the active noise control system 26 will effectively counteract undesirable noise in response to the ANC controller 34 as generally known.

Referring to FIG. 2, the speaker 30 is located within an enclosure 36. The enclosure 36 is mounted within the vehicle body component 16 typically forward of a vehicle wheel well 38 (FIG. 3). The enclosure 36 is a distance from the vehicle passenger compartment 40. As generally known a firewall 42 separates the vehicle engine compartment 44 from the passenger compartment 40. An elongated transmission member 46 preferably communicates canceling noise from the speaker 30 over the wheel well 38 to an outlet 48 adjacent the firewall 42. The elongated transmission member 46 focuses and directs cancellation noise toward the firewall 42. The elongated transmission member 46 also bypasses the wheel well and any restriction created thereby.

The speaker enclosure 36 includes an enclosure outlet 50 which communicates with an inlet 51 to the elongated transmission member 46. It should be understood that an enclosure of any size in shape will benefit from the present invention, however, it is preferred that a relatively sealed cavity be provided to enhance noise transmission and protect components from the environment.

The elongated transmission member 46 is preferably a tubular member having a flared outlet 48. The outlet 48 is located a distance from the firewall 42. That is, the outlet 48 need not penetrate the firewall 42. Installation is thereby simplified. Moreover, no structural weakness is introduced.

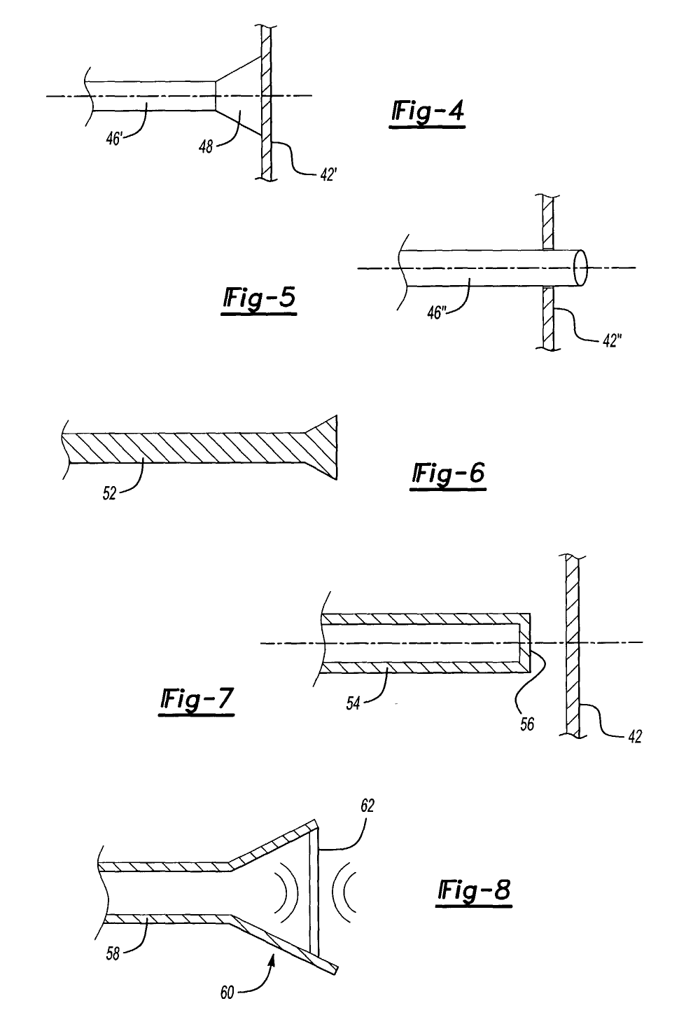

Alternatively, the elongated transmission member 46′ may contact the firewall 42′ (FIG. 4) or the elongated transmission member 46″ may pass through the firewall 42″ (FIG. 5).

Referring to FIG. 6, the elongated transmission member 52 may alternatively be a solid member that transmits cancellation noise through resonance thereof.

Referring to FIG. 7, an elongated transmission member 54 may alternatively be a hollow member having a closed end 56 which transmits cancellation noise through resonance thereof. Although the end 56 adjacent the firewall 42 is illustrated as the closed end in the illustrated embodiment, it should be understood that either end may be closed. Moreover, the end 56 may contact or penetrate the firewall 42.

Referring to FIG. 8, an elongated transmission member 58 may alternatively be a hollow member having a closed end 60 having a flexible membrane 62 therein to amplify the cancellation noise through resonance thereof.

Depending upon the desired the desired cancellation noise various alternatives to a completely hollow elongated transmission member will benefit from the present invention. Moreover, various combinations of the disclosed arrangements will benefit from the present invention depending upon the desired cancellation noise.

The foregoing description is exemplary rather than defined by the limitations within. Many modifications and variations of the present invention are possible in light of the above teachings. The preferred embodiments of this invention have been disclosed, however, one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

What is claimed is:

1. An active noise control system for attenuating noise from a vehicle comprising:
an active noise cancellation speaker mounted remote from a vehicle firewall;
an elongated transmission member to transmit cancellation noise from said active noise cancellation speaker toward said vehicle firewall.

2. The active noise control system as recited in claim 1, wherein said elongated transmission member comprises a hollow member.

3. The active noise control system as recited in claim 1, wherein said elongated transmission member comprises a flared end.

4. The active noise control system as recited in claim 1, wherein said elongated transmission member comprises a solid member.

5. The active noise control system as recited in claim 1, wherein said elongated transmission member comprises a closed end.

6. The active noise control system as recited in claim 1, further comprising a flexible membrane within an end of said elongated transmission member.

7. The active noise control system as recited in claim 1, further comprising an enclosure, said active noise cancellation speaker mounted within said enclosure.

8. The active noise control system as recited in claim 7, wherein said enclosure communicates with said elongated transmission member.

9. The active noise control system as recited in claim 1, wherein an end of said elongated transmission member penetrates said vehicle firewall.

10. The active noise control system as recited in claim 1, wherein an end of said elongated transmission member contacts said vehicle firewall.

11. The active noise control system as recited in claim 1, wherein said elongated transmission member passes adjacent a vehicle wheel well.

12. A vehicle body component having a wheel well comprising:
an enclosure mounted remote from a vehicle firewall; and
an active noise cancellation speaker mounted within said enclosure;
an elongated transmission member which passes adjacent said wheel well to transmit cancellation noise from said active noise cancellation speaker toward said vehicle firewall.

13. The vehicle body component as recited in claim 12, wherein said elongated transmission member comprises a hollow member.

14. The vehicle body component as recited in claim 12, wherein said elongated transmission member comprises a flared end.

15. The vehicle body component as recited in claim 12, wherein said elongated transmission member comprises a solid member.

16. The vehicle body component as recited in claim 12, wherein said elongated transmission member comprises a closed end.

17. The vehicle body component as recited in claim 12, further comprising a flexible membrane within an end of said elongated transmission member.
